(19) United States
(12) Patent Application Publication
(10) Pub. No.: US 2007/0020194 A1
(43) Pub. Date: Jan. 25, 2007

(54) Nasally administered appetite suppressant

(76) Inventors: Frank Lyons Greenway III, Baton Rouge, LA (US); Linda Robin De Young, Montara, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/449,417

(22) Filed: Jun. 8, 2006

Related U.S. Application Data
(60) Provisional application No. 60/688,907, filed on Jun. 8, 2005.

(51) Int. Cl. A61L 9/04; A61K 31/4745; A61K 31/4166; A61K 31/137; A61K 31/24

(52) U.S. Cl. 424/45; 514/305; 514/389; 514/536; 514/649

(57) ABSTRACT

Methods are described for suppressing appetite through the intranasal administration of a sodium channel blocker. It has long been known that smell and taste are an important part of how the body prepares for a meal, for example the cephalic phase of insulin release before a meal. Depressing the sense of smell or taste in subjects prior to meals leads to decreased food intake, which ultimately leads to weight loss. The ability to deliver drugs to the CSF via intranasal application also provide anorexiant central effect.

### PRIORITY CLAIM

This application claims priority under 35 U.S.C. § 119(e) to U.S. Provisional Application Ser. No. 60/688,907, filed Jun. 8, 2005, entitled “Nasally Administered Appetite Suppressant,” which is incorporated herein by reference in its entirety.

### BACKGROUND OF THE INVENTION

1. Field of the Invention

The present invention is in the field of pharmaceutical compositions for the treatment of obesity and for affecting weight loss in individuals by intranasal delivery of sodium channel blockers through both inhibition of olfactory neurosensory perception and centrally mediated effects via direct transport of sodium channel blockers into the cerebral spinal fluid (“CSF”).

2. Description of the Related Art

Obesity is a disorder characterized by the accumulation of excess fat in the body. Obesity is emerging as a global problem and is a major factor for a number of co-morbidities such as coronary heart disease, hypertension, non-insulin dependent diabetes mellitus, pulmonary dysfunction, osteoarthritis and certain types of cancer.

Obesity has been defined in terms of body mass index (BMI). BMI is calculated as weight (kg)/[height (m)]2. In addition to those individuals who satisfy a strict definition of medical obesity, a significant portion of the adult population is overweight. These individuals would also benefit from the availability of an effective weight-loss composition. Current products to suppress appetite and control weight are generally drugs with undesirable side effects. The main factor causing the development of obesity is a positive energy balance through the decreased activity and increased energy intake. Weight loss and loss of body fat can thus be achieved by reducing food intake and/or increasing energy expenditure.

Studies show that weight tends to decline after a certain age. The reason for the decline in weight with aging has been attributed to the normal decline in the taste and smell senses. The smell of food alone has been demonstrated to increase pancreatic polypeptide within the first 3 minutes and to increase colonic pressure. The sight and smell of food increase insulin secretion in the first 20 minutes and this rise in insulin is blocked by atropine, suggesting that the rise is vagally mediated. Patients with anorexia have been shown to have a diminished sense of smell and in case reports, a diminished sense of taste and smell have been associated with weight loss. The smell of food also increases appetite and food intake in restrained eaters. Smell and taste are perceived by a multitude of G-protein coupled receptors expressed on specialized nerve cells in the oral and nasal epithelium.

Also, the neural connections between the nasal mucosa and the brain provide a unique pathway for noninvasive delivery of therapeutic agents to the central nervous system (“CNS”).

### SUMMARY OF THE INVENTION

One embodiment of the invention is a method of treating obesity in a mammal in need of such treatment including applying a sodium channel blocker to the mammal intranasally in an amount sufficient to decrease sensation of smell.

In some preferred embodiments of the invention, the sodium channel blocker may be lidocaine.

In some embodiments, the sodium channel blocker is applied before meals and may be in the form of a gel, powder, spray, liquid or drop.

In other embodiments, a vasoconstrictor is also administered. The vasoconstrictor can be, for example, epinephrine, norepinephrine, endothelin, thromboxane, naphazoline nitrate, tetrahydrozoline hydrochloride, oxymetazoline hydrochloride, phenylephrine hydrochloride and tramazoline hydrochloride.

In one embodiment, the concentration of the sodium channel blocker may be from about 0.05 mg/ml to about 200 mg/ml.

In some preferred embodiments, the sodium channel blocker may be selected from lidocaine, mepivacaine, bupivacaine quinidine, lorcainide, procainamide, encainide, propafenone, moricizine, mexiletine, disopyramide, aprindine, phenytoin, tocainide, flecainide, procaine, benzocaine dibucaine, tetracaine, butacaine, cyclomethycaine and tetracaine.

In another embodiment, a topical anesthetic may be co-administered to the oral mucosa or tongue to reduce taste sensation.

Another embodiment of the invention is a method of treating obesity in a mammal including applying a sodium channel blocker, a humectant, a pH buffer and a thickening agent to the mammal intranasally.

In a preferred embodiment, the humectant may be selected from sorbitol, mineral oil, vegetable oil, glycerol, soothing agents, membrane conditioners, sweeteners and combinations thereof.

In another preferred embodiment, the pH buffer can be selected from acetate, citrate, prolamine, carbonate and phosphate buffers.

In another preferred embodiment, the thickening agent may be selected from xanthan gum, carboxymethyl cellulose, hydroxypropyl cellulose and carbomer

### DETAILED DESCRIPTION OF THE INVENTION

The effect that the smell of food has on the body and appetite is well documented. Therefore, temporarily depressing the sense of taste and smell prior to meals may be an effective strategy to decrease food intake. Embodiments of the invention relate to methods of appetite suppression through the inhibition of olfactory neurosensory perception. In one embodiment, a sodium channel blocker is applied intranasally to a mammal in need of weight loss. Thus, in one exemplary embodiment of the invention, lidocaine, a widely used topical anesthetic which has the ability to temporarily disable nerve conduction, can be applied to the nostrils of a subject before a meal. Lidocaine applied topically in the nose decreases the sense of smell by altering the permeability of the olfactory cells to ions and inhibiting olfactory nerve impulses. Lidocaine may also be directly taken up in the CSF and have a central effect on satiety and food consumption. Some of the weight loss effects of the sodium channel blockers may be due to regional activity in the brain. The olfactory neural pathway provides both intraneuronal and extraneuronal pathways into the brain. The intraneuronal pathway involves axonal transport and requires hours to days for drugs to reach different brain regions. The extraneuronal pathway probably relies on bulk flow transport through perineural channels, which deliver drugs directly to the parenchymal tissue of the brain, to the cerebrospinal fluid (CSF), or to both. This extraneuronal pathway allows therapeutic agents to reach the CNS within minutes. Intranasal delivery of agents to the CSF is not surprising as CSF normally drains along the olfactory axon bundles as they traverse the cribriform plate of the skull and approach the olfactory submucosa in the roof of the nasal cavity where the CSF is then diverted into the nasal lymphatics. With reduced olfactory perception of the food in the meal and reduced appetite, the subject exhibits a reduced physiological response which results in lower food intake.

Sodium channel blockers are preferred over other currently available weight loss drugs because of reduced side effects. Many weight loss drugs currently sold have many negative side effects which range from merely unpleasant to hazardous. Sodium channel blockers, on the other hand, have very few side effects and have been used with relatively little risk for many decades. Lidocaine, for example, applied topically gives peak anesthesia in 2-5 minutes that lasts for 30-45 minutes. The maximum safe dosage for topical anesthetic in a 70 kg adult is extremely high, 500 mg, and the intravenous infusion rate following a loading bolus of 50-100 mg is 72 mg in a 24-hour period. Therefore, doses of a sodium channel blocker such as lidocaine, for example, in amounts up to 100 mg per nostril per day would be well within the safe range for topical treatment. Furthermore, sodium channel blockers are advantageous over other agents applied intranasally due to the lower incidence of nasal mucus membrane irritation.

Other sodium channel blockers that can be used in the present invention include, for example, mepivacaine, bupivacaine quinidine, lorcainide, procainamide, encainide, propafenone, moricizine, mexiletine, disopyramide, aprindine, phenytoin, tocainide flecainide, procaine, benzocaine dibucaine, tetracaine, butacaine, cyclomethycaine and tetracaine. In a preferred embodiment, the localized effect of these anesthetics is enhanced and prolonged by combining them with a vasoconstrictor in a manner known in the art. Vasoconstrictive agents are added to local anesthetics for reduction of plasma concentration of local anesthetics, reduction of minimal concentration of local anesthetic necessary for arresting the nerve impulse and prolonged effect of local anesthesia.

Suitable vasoconstrictors include, for example, epinephrine, norepinephrine, endothelin, thromboxane, naphazoline nitrate, tetrahydrozoline hydrochloride, oxymetazoline hydrochloride, phenylephrine hydrochloride, tramazoline hydrochloride, levonordefrin (Neo-Cobefrine) and nordefrin (Cobefrine).

Any therapeutic dosage, one that achieves at least a perceptible blockage of olfactory sensation, or elicits the central effects needed for appetite suppression, is appropriate and is considered to be within the scope of the present invention. In a preferred embodiment, lidocaine is provided in nasal drops, gel, powder, spray, or instillation liquid in a concentration of from about 0.05 to about 200 mg/ml, preferably about 20 mg/ml.

In the method of the present invention, the anesthetic is delivered into the nasal passageways in any suitable manner, such as by instillation of a liquid, drops, a gel, a powder or a nasal spray. This is preferably done at least a few minutes (e.g., 1, 2, 5, 10, 15 or more) prior to a meal or other consumption of food, thereby allowing the anesthetic to take action. Because some of the anesthetics of the present invention are short-acting, it may be desirable to repeat the treatment after a suitable period, e.g., after 15, 30, 45, or 60 minutes. With some of the central acting effects, it may be possible to reduce frequency of administration.

The various forms of the delivery system set forth above can include a buffer to maintain the pH of the sodium channel blocker and a pharmaceutically acceptable thickening agent and a humectant. Desirably, the pH of the buffer is selected to maintain the sodium channel blocker in a non-ionized form. In particular, the pH of the buffer is selected to optimize the absorption of the sodium channel blocker across the nasal mucosa. The particular pH of the buffer can vary depending upon the particular nasal delivery formulation as well as the specific sodium channel blocker composition selected. Examples of buffers suitable for use in the present invention include acetate, citrate, prolamine, carbonate and phosphate buffers. With respect to the non-aqueous formulations set forth above, suitable forms of buffering agents can be selected such that when the formulation is delivered into the nasal cavity of a mammal, selected pH ranges are achieved therein upon contact with, e.g., a nasal mucosa. In the present invention, the pH of the compositions should be maintained from about 3.0 to about 10.0. Compositions having a pH of less than about 3.0 or greater than about 10.0 can increase the risk of irritating the nasal mucosa of a recipient. Further, it is preferable that the pH of the compositions be maintained from about 3.0 to about 7.0.

Viscosity of the compositions may be maintained at the selected level using a therapeutically acceptable thickening agent. In one embodiment, methyl cellulose is used because it is readily and economically available and is easy to work with. Examples of other suitable thickening agents include xanthan gum, carboxymethyl cellulose, hydroxypropyl cellulose and carbomer. The preferred concentration of the thickener will depend upon the agent selected. Viscous compositions are normally prepared from solutions by the addition of such thickening agents.

The compositions of the present invention can also include a humectant to reduce or prevent drying of the mucus membrane and to prevent irritation thereof. Examples of humectants that can be used in the present invention include sorbitol, mineral oil, vegetable oil and glycerol. The concentration of the humectant in the present compositions will vary depending upon the agent selected. Also, soothing agents, membrane conditioners, sweeteners and combinations thereof can be included in the present invention.

In other embodiments of the invention, other optional ingredients can also be incorporated into the nasal delivery system provided that they do not interfere with the action of the sodium channel blocker or significantly decrease the absorption of the sodium channel blocker across the nasal mucosa. Such ingredients include pharmaceutically acceptable excipients and preservatives.

In some embodiments, preservatives are also included in the present invention. Examples of preservatives that can be used with the present invention include benzyl alcohol, parabens, thimerosal, chlorobutanol and benzalkonium chloride and preferably benzalkonium chloride. Typically, the preservative will be present in a composition in a concentration of up to about 2% by weight. The exact concentration of the preservative, however, will vary depending upon the intended use and can be ascertained by one skilled in the art.

In some embodiments of the invention, therapeutically acceptable surfactants are included. Examples of suitable surfactants include polyoxyethylene derivatives of fatty acid partial esters of sorbitol anhydrides such as Tween 80, Polyoxyl 40 Stearate, Polyoxyethylene 50 Stearate and Octoxynol. The usual concentration is from about 1% to about 10% based on the total weight.

In other embodiments of the invention, bioadhesive agents can be used to increase nasal absorption. These compounds promote binding of drugs to biological material in the nasal cavity, thereby extending residence times and allowing increased absorption. Examples of bioadhesive materials include carbopol, cellulose agents, starch, dextran, and chitosan. Those skilled in the art will recognize that the components of the compositions are advantageously selected to be chemically inert with respect to the active agent.

Another aspect of the present invention includes coadministration of the nasal preparation with an oral preparation, in order to simultaneously decrease both smell and taste sensations. The oral preparation can advantageously utilize one of the same sodium channel anesthetics used in the nasal preparation, or a different one, and can take any suitable oral delivery form, e.g., drops, creams, powders, gels, lozenges, fast melt strips, and liquids. The concentration and dosage of the oral form is substantially similar to that of the nasal preparation, except that, if desired, the oral dosage can be greater than the nasal dosage (e.g., 1, 2, 4, or more times the nasal dosage).

As with the nasal preparation, a vasoconstrictor can also advantageously be used with the oral form. It should be noted that various oral anesthetic preparations containing topical anesthetic are commercially available, and include gels, mouthwash, and lozenges, all of which can be used in the present invention.

Certain aspects of the invention are exemplified in more detail in the following examples:

### EXAMPLE 1

Three males and one female between 23 and 60 years of age with BMI's less than 25 kg/m2 participated in a study of food intake. The subjects presented to the food intake laboratory at noon after an overnight fast except for water from 9 pm the prior night on two occasions a week apart. They had 10 mg (0.5 cc) of lidocaine or 0.5 cc normal saline administered in each nostril 20 minutes before presentation of a meal consisting of fried chicken pieces and water. They were allowed to eat and drink as much or as little as they wished over a 20-minute period, and there were more chicken pieces than they could reasonably be expected to eat at one sitting. The order of the lidocaine and saline was balanced and the study was performed in a double-blind fashion.

The four subjects consumed 15%, 11%, 27% and 33% less on the lidocaine week compared to the placebo week. The greatest reductions were seen in the 23-year-old woman (33%) and 59-year-old male (27%). The overall reduction in food intake 21.5%+10.2% was statistically significant (p<0.02). Sibutramine, an obesity drug, gives an 8% weight loss at 6 months and causes a 12% decrease in food intake. The results of this pilot study suggest that if the food intake reduction is sustained, intranasal lidocaine might result in as much as a 14% weight loss.

### EXAMPLE 2

Thirty healthy subjects 18 years of age or older with a BMI between 30-40 and on no regular medications except contraceptives or hormone replacement therapy participate in a trial. At lunch-time on 4 separate occasions they receive 0.5 cc of fluid in each nostril and complete their food intake tests. Participants are instructed not to eat or drink anything 12 hours before each test meal. A meal of fried chicken pieces in an amount larger than could reasonably be consumed begins approximately 5 minutes after the nose drops are administered. The participant are allowed to use a condiment with the chicken. Participants are allowed to eat as much or as little as they wish. Laboratory staff record the duration (seconds) of the meal. Eating behavior is monitored by the weight of the food consumed and/or by video tape/camera. Subjects complete a set of Visual Analogue Scales (VAS) to measure appetite before the nose drops, and before and after each meal. Questions to assess the sensory properties of the chicken are administered after the nose drops have been given, but prior to each meal. All subjects receive the following in a balanced order and in a double-blind fashion: saline, 2.5 mg lidocaine per nostril, 10 mg lidocaine per nostril, and 25 mg lidocaine per nostril in 0.5 cc of fluid. Food intake is monitored by the Universal Eating Monitor (UEM). The UEM consists of a scale that is concealed in a table and connected to a computer, which automatically records the weight of food removed (eaten) from a plate on top of the scale. The table is covered with a tablecloth and the research participant is not acutely aware food intake is being monitored. The UEM records the cumulative amount of food consumed and the time at which each bite of food is eaten. Therefore, a curve is generated by plotting the cumulative food intake on the y-axis against time units on the x-axis. This curve displays a person's rate of food intake and the reduction in food consumption via escalating doses of lidocaine.

### EXAMPLE 3

The procedure of Example 2 is repeated, except that about 0.5 cc of 0.25% to 0.5% phenylephrine nasal formulation is sprayed in each nostril in addition to the 0.5 cc of the lidocaine and saline solution. The phenylephrine prolongs the half-life of the lidocaine in vivo.

### EQUIVALENTS

The foregoing description details certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A method of treating obesity in a mammal in need of such treatment comprising applying a sodium channel blocker to the mammal intranasally in an amount sufficient to decrease sensation of smell.

2. The method of claim 1, wherein the sodium channel blocker is lidocaine.

3. The method of claim 1, wherein the sodium channel blocker is applied before meals.

4. The method of claim 1, wherein the sodium channel blocker is in the form of a gel, powder, spray, liquid, or drop.

5. The method of claim 1, further comprising the administration of a vasoconstrictor in combination with the sodium channel blocker.

6. The method of claim 5, wherein the vasoconstrictor is selected from the group consisting of: epinephrine, norepinephrine, endothelin, thromboxane, naphazoline nitrate, tetrahydrozoline hydrochloride, oxymetazoline hydrochloride, phenylephrine hydrochloride and tramazoline hydrochloride.

7. The method of claim 1, wherein the concentration of the sodium channel blocker is from about 0.05 to about 200 mg/ml.

8. The method of claim 1, wherein the sodium channel blocker is selected from the group consisting of: lidocaine, mepivacaine, bupivacaine quinidine, lorcainide, procainamide, encainide, propafenone, moricizine, mexiletine, disopyramide, aprindine, phenytoin, tocainide, flecainide, procaine, benzocaine dibucaine, tetracaine, butacaine, cyclomethycaine and tetracaine.

9. The method of claim 1, further comprising the co-administration of a topical anesthetic to the oral mucosa or tongue to reduce taste sensation.

10. A method of treating obesity in a mammal in need of such treatment comprising applying a sodium channel blocker, a humectant, a pH buffer and a thickening agent to the mammal intranasally.

11. The method of claim 10, wherein the humectant is selected from sorbitol, mineral oil, vegetable oil, glycerol, soothing agents, membrane conditioners, sweeteners and combinations thereof.

12. The method of claim 10, wherein the pH buffer is selected from acetate, citrate, prolamine, carbonate and phosphate buffers.

13. The method of claim 10, wherein the thickening agent is selected from xanthan gum, carboxymethyl cellulose, hydroxypropyl cellulose and carbomer.
